(19) United States
(12) Patent Application Publication
(10) Pub. No.: US 2025/0127410 A1
(43) Pub. Date: Apr. 24, 2025

(54) WHOLE-BODY PHYSICAL FATIGUE MONITORING WITH HEART RATE

(71) Applicant: The Regents of the University of Michigan, Ann Arbor, MI (US)

(72) Inventors: Juhyeon Bae, Ann Arbor, MI (US); Gaang Lee, Edmonton (CA); SangHyun Lee, Ann Arbor, MI (US)

(21) Appl. No.: 18/490,688

(22) Filed: Oct. 19, 2023

(51) Int. Cl. A61B 5/024; A61B 5/00; A61B 5/16; G09B 23/28; G16H 15/00; G16H 50/20; G16H 50/30

(52) U.S. Cl. CPC A61B 5/02416; A61B 5/165; A61B 5/4866; A61B 5/681; A61B 5/6824; A61B 5/7246; A61B 5/7275; A61B 5/7282; A61B 5/7405; A61B 5/742; A61B 5/7455; A61B 5/746; A61B 5/7475; G09B 23/28; G16H 15/00; G16H 50/20; G16H 50/30; A61B 5/0022; A61B 2503/20; A61B 2560/0223; A61B 2560/0462

(57) ABSTRACT

The current disclosure provides systems and methods for monitoring a whole-body fatigue (WBF) of a worker while the worker performs physical activity (e.g., labor) via a wearable WBF assessment device worn by the worker. The WBF is calculated using a critical power (CP) bioenergetic model that is individually calibrated to the worker based on periodic WBF self-assessments performed by the worker via the WBF assessment device. The WBF assessment device includes a biosensor that monitors the worker's heart rate. The customized CP model is based on estimating a physical intensity of the physical activity based on a percentage of heart rate reserve (% HRR) of the worker calculated from the heart rate.

### TECHNICAL FIELD

Embodiments of the subject matter disclosed herein relate to monitoring a level of physical fatigue in a human body.

### BACKGROUND

Fieldwork often involves heavy workloads, prolonged work hours, and harsh work environments, such as working outdoors in hot and humid weather. These physically demanding work conditions naturally predispose workers to physical fatigue. Physical fatigue is closely associated with impaired physical and cognitive capabilities as well as increased unsafe behavior, leading to safety and productivity issues. To manage field workers physical fatigue, various efforts have been undertaken to develop techniques to monitor workers' physical fatigue during ongoing work in the field.

However, most existing physical fatigue monitoring techniques applicable in the field aim to monitor localized muscle fatigue rather than whole-body fatigue (WBF). WBF commonly occurs in field jobs when workers engage in taxing whole-body activities for extended periods. Unlike localized muscle fatigue, whole-body fatigue triggers a defensive neuro-physiological mechanism that reduces workers' bodily activation and alertness, thereby slowing down their perception and cognition. As a result, workers exhibit impaired abilities to react to surrounding hazards, potentially leading to accidents.

Some existing field-applicable solutions focus on assessing WBF based on a heart rate of a worker, as measured by a wearable photoplethysmogram (PPG) sensor worn by the worker, for example, on a wrist of the worker. A bioenergetic model may be used to estimate the WBF of the worker based on the heart rate. However, current solutions may not be applicable at scale in practice, because an assessment of WBF may vary widely across different individuals. As a result, generating a reliable measurement of WBF for a particular worker may involve calibrating the bioenergetic model to the particular worker. The model may be calibrated by administering periodic field surveys to the worker. However, getting sufficient high quality survey data may be difficult, because administering the surveys may interrupt a task being performed by the worker. The worker may find the surveys distracting or annoying, or the workers may not be able to respond to a survey during certain tasks.

### SUMMARY

The current disclosure at least partially addresses one or more of the above identified issues by a method for a WBF assessment device used to assess a whole-body fatigue (WBF) of a worker, the method comprising collecting heart rate data of the worker via a biosensor of the WBF assessment device; estimating the WBF of the worker based on the collected heart rate data, using a bioenergetic model; and in response to the WBF exceeding a threshold WBF, notifying the worker; wherein the bioenergetic model is individually calibrated to the worker based on a plurality of WBF self-assessments performed by the worker via the WBF assessment device at regular time intervals while the worker is performing physical activity (e.g., labor). The WBF assessment device may be a wearable device worn on a wrist of the worker, which may periodically request that the user perform a WBF self-assessment, for example, by displaying a message on a screen of the WBF assessment device, or via an audio or haptic signal. The WBF self-assessment may comprise a rating of fatigue (ROF) based on a pictographic single-item numerical scale that can be performed by the worker via the WBF assessment device in less than 15 seconds during the physical activity, with minimal interruption of work.

In this way, the WBF assessment device enables scalable WBF monitoring for field workers by utilizing an affordable and simple wristband-type biosensor in conjunction with the bioenergetic model. The bioenergetic model explains how physical activities at the whole-body level accumulate whole-body fatigue. Specifically, workers' physical intensity is measured from heart rate data collected by a wristband. The proposed technique creates a regression model by conducting a brief individual calibration, in order to estimate a physical intensity threshold that determines whether WBF is accumulating or decumulating, as well as a slope between physical intensity and WBF for each individual. The individual regression model is then utilized to monitor the level of WBF from the measured physical intensity. By doing so, preventive interventions for managing field workers' WBF can be implemented before serious detrimental outcomes, such as physical exhaustion, cognitive error-related accidents, or other accidents that occur from prolonged physical activities, contributing to advancing the safety and productivity of field workers.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a block diagram illustrating an overall process for assessing a WBF of a worker, in accordance with one or more embodiments of the present disclosure;

FIG. 2 is a block diagram showing an exemplary WBF assessment device worn by the worker, in accordance with one or more embodiments of the present disclosure;

FIG. 3 is a flowchart showing an exemplary high-level method for assessing a WBF of the worker, in accordance with one or more embodiments of the present disclosure;

FIG. 4 is a flowchart showing an exemplary method for collecting a self-assessment from the worker via the WBF assessment device, in accordance with one or more embodiments of the present disclosure;

FIG. 5 is a flowchart showing an exemplary method for calculating the WBF of the worker based on heart rate data and self-assessment data collected from the worker, in accordance with one or more embodiments of the present disclosure;

FIG. 6 is a diagram illustrating how the WBF of the worker is calculated, in accordance with one or more embodiments of the present disclosure; and

FIG. 7 shows an exemplary pictograph based on the numerical scale, used by the worker to rate their WBF in a self-assessment, in accordance with one or more embodiments of the present disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems, and methods.

### DETAILED DESCRIPTION

Field work in industries like construction, agriculture, oil, and mining often involves heavy workloads, prolonged hours, and harsh work environments (e.g., hot weather). These conditions predispose field workers to physical fatigue, defined as muscles' inability to maintain required strength during work, involving a feeling of tiredness. Fatigue in field work is problematic given its negative impact on workers' productivity, safety, and health. Physical fatigue coincides with demotivation, poor judgment, and inattentiveness, affecting work quality and job satisfaction. In addition, physical fatigue reduces physical ability, impairs cognition, and promotes unsafe behavior. It should be appreciated that physical fatigue as described herein does not cover chronic fatigue observed in individuals with medical or psychiatric conditions.

In practice, assessing physical fatigue has relied on workers' subjective responses to fatigue scales, such as the Swedish Occupational Fatigue Inventory (SOFI), Occupational Fatigue Exhaustion Recovery Scale, and Fatigue Assessment Scale for Construction Workers (FASCW). However, such surveys are conducted sporadically and may demand that workers stop their ongoing work to participate. As a result, such approaches may be limited in understanding the dynamics of physical fatigue during ongoing work, reducing the potential for timely, proactive managerial or worker intervention.

Field work commonly involves two main types of acute physical fatigue: localized muscle fatigue (LMF) and whole-body fatigue (WBF). LMF is characterized by fatigue that is limited to specific muscle groups, while WBF occurs by a prolonged whole-body activity and affects the entire body. LMF is common in tasks requiring high and repetitive force generation on specific muscle groups, such as drilling and sewing machine operation. LMF can cause decreased physical performance and aching and soreness in the affected muscle groups, thereby negatively affecting workers' productivity and increasing the risk of developing work-related musculoskeletal disorders.

Unlike LMF, WBF involves the entire body during physically demanding activities. The mechanisms behind WBF are not fully understood but involve a complex interplay between peripheral (muscular) and central (neural) factors. This interaction of central and peripheral mechanisms explains why WBF can occur during many field tasks involving whole-body actions, despite the limited use of individual muscle groups.

WBF can be more significant than LMF as the WBF's central factors trigger a defensive neuro-physiological mechanism to maintain homeostasis, whereby the central nervous system reduces the levels of bodily activation, thereby decreasing workers' alertness—slowing down their perception and cognition. Consequently, workers experiencing WBF tend to show an impaired ability to react to surrounding hazards in the field, potentially leading to accidents and impacting workers' safety and productivity.

To overcome the limitations of a sporadic and invasive questionnaire-based fatigue assessment, some approaches have relied on wearable biosensors for continuous and less invasive fatigue assessment during daily activities. For instance, wearable electromyography (EMG) sensors have been applied to detect fatigued muscles. However, such efforts have focused on LMF, not WBF.

Studies exploring the use of wearable biosensors in understanding workers' WBF have demonstrated the usefulness of biosignals collected from wearables in assessing WBF. The techniques used typically detect physical fatigue in workers by monitoring symptoms related to increased physical exertion (e.g., changes in heart rate, heart rate variability or skin temperature), assuming a consistent correlation between WBF and physical exertion. However, this assumption is validated only when workers undergo constant physical demand. In a naturalistic work environment where workers often take rests and physical demand changes irregularly, the relationship between WBF and physical exertion may not be consistent. If a worker's physical intensity changes, the worker's exertion may follows the change in intensity, rather than indexing changes in WBF. For example, a worker may transition from a more physically demanding task to a less physically demanding task (e.g., decreased physical intensity). When the worker's intensity decreases, the worker's exertion may decrease, while the worker may still suffer from a high level of WBF as a result of the time spent on the more physically demanding task. Similarly, during a break after an excessively demanding physical activity, the worker may still experience a certain level of WBF, but not perceive any exertion. Therefore, there is a need for a new wearable biosensor-based technique that can assess WBF in physical demand-varying naturalistic work environments.

In some approaches, when workers' WBF is monitored during work with a wrist-worn biosensor, a critical power (CP) model may be applied, as described in greater detail below. The CP model is a widely validated bioenergetic model, and in a naturalistic work environment characterized by frequent rests and natural physical demand fluctuations, CP model-based techniques may outperform alternative physical exertion-based approaches that rely on biosignal metrics such as skin temperature, heart rate, standard deviation of the normal heartbeat intervals (SDNN), root mean square of successive differences between normal heartbeats (RMSSD), low vs. high frequency ratio in blood volume pressure (LF/HF), and/or others.

The CP model assumes that there are two energy supplies for human physical activities: aerobic and anaerobic supplies. It is also assumed that aerobic energy's supply capacity [kJ] is unlimited but that its rate is limited, while anaerobic work capacity [kJ] is limited but is not rate-limited. Here, the rate limit of aerobic work (e.g., an aerobic work threshold) is referred to herein as a CP threshold or CP. Therefore, when a level of physical intensity (PI) of the physical activity is less than the CP, the aerobic supply's rate limit, we can theoretically expect that the person can continue the ongoing physical activity infinitely depending on their aerobic supply. On the other hand, when the PI is more than the CP, the physical activity requires the use of anaerobic energy supply. Since anaerobic work capacity is limited, if the physical activity continues, the person eventually uses up all their anaerobic work capacity and ends up being physically exhausted. Thus, the level of WBF can be expressed as the proportion of expended anaerobic work capacity over its total anaerobic work capacity, and physical exhaustion is defined as 100% WBF, where anaerobic work capacity is totally depleted.

However, there is a hurdle in applying the CP model to measure the WBF in workplaces. Unlike sport exercise where PI can be relatively easily measured by observing the output performance of repeated activities such as a running and cycling pace, in a workplace it can be challenging to continuously monitor variable PI during tasks that are less repeated and involve diverse forms of physical activity. Other approaches to monitoring WBF using the CP model have not suggested a practical way to track variable PI in workplaces, which is essential for effective WBF monitoring. An additional drawback of WBF monitoring using the CP model that has not been successfully addressed is that the CP model does not take into account variability between individuals. Specifically, the CP model includes factors that vary based on age, weight, height, and other health conditions. As a result, accurate WBF monitoring of a particular individual may rely on calibrating the CP model for the particular individual.

To address these issues, a minimally invasive wearable WBF assessment device is proposed herein that may be used to both monitor a worker's heart rate and collect periodic self-assessments of the worker's WBF from the worker, which may be used to calibrate the CP model for more accurate WBF monitoring. The WBF assessment device may facilitate collection of the heart rate data and the self-assessment data in a way that minimizes an interruption of work performed by the worker.

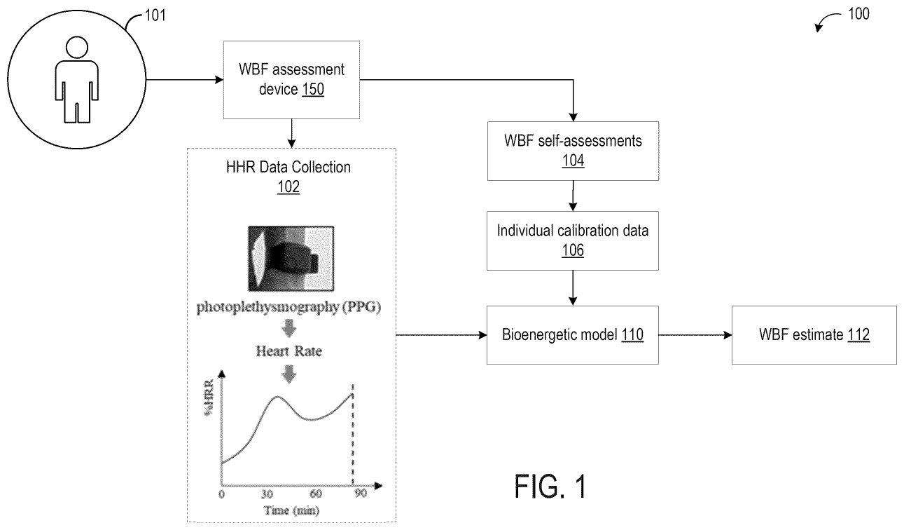

FIG. 1 shows a high-level procedure 100 for monitoring a WBF of a worker 101 during physical activity. The physical activity may vary with respect to a physical intensity (PI), which may be low or high. For example, the worker may perform a variety of tasks in series, where some of the tasks are lower PI tasks, and some of the tasks are higher PI tasks. While performing the tasks, the worker may undergo periods of higher exertion (e.g., during the higher PI tasks) and periods of lower exertion (e.g., during the lower PI tasks).

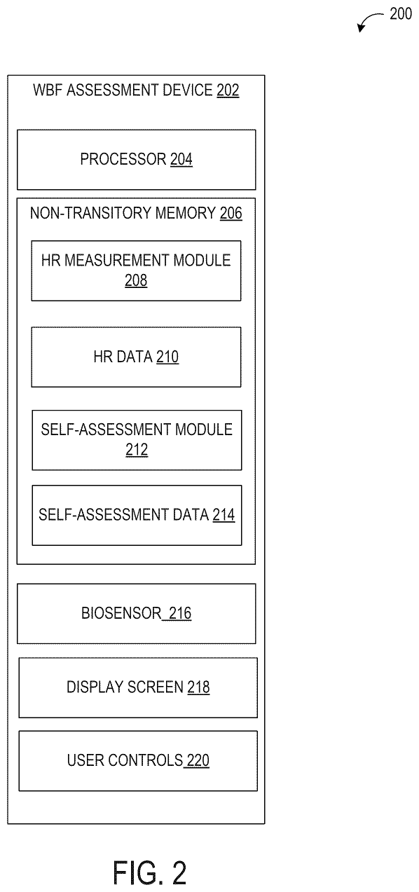

The WBF of worker 101 may be monitored by a wearable WBF assessment device 150 worn by worker 101 while performing the physical activity. An exemplary WBF assessment device is described below in reference to FIG. 2. In various embodiments, WBF assessment device 150 may be worn on a wrist of the worker.

During a heart rate data collection stage 102, a heart rate (HR) of the worker may be monitored by a biosensor of WBF assessment device 150. In various embodiments, the biosensor measures photoplethysmography (PPG) at the wrist of the worker. PPG is an optical signal acquired by illuminating skin and monitoring changes in the skin's light absorption, reflection, and scattering. Changes in the absorbed or reflected light are detected by a photodetector and used to calculate heart activity-related metrics, such as HR and HR variability. The HR data may be used to calculate worker 101's percentage of heart rate reserve (% HRR), where HRR is the difference between a maximum or peak HR of worker 101 (e.g., during a highest PI task) and a resting HR of worker 101 (e.g., when worker 101 is at rest), and % HRR is the difference between a resting HR and a current HR divided by the HRR. The % HRR may be an indicator of PI, where % HRR may be used by a bioenergetic model 110 (e.g., the CP model described above) to generate a WBF estimate 112 of worker 101.

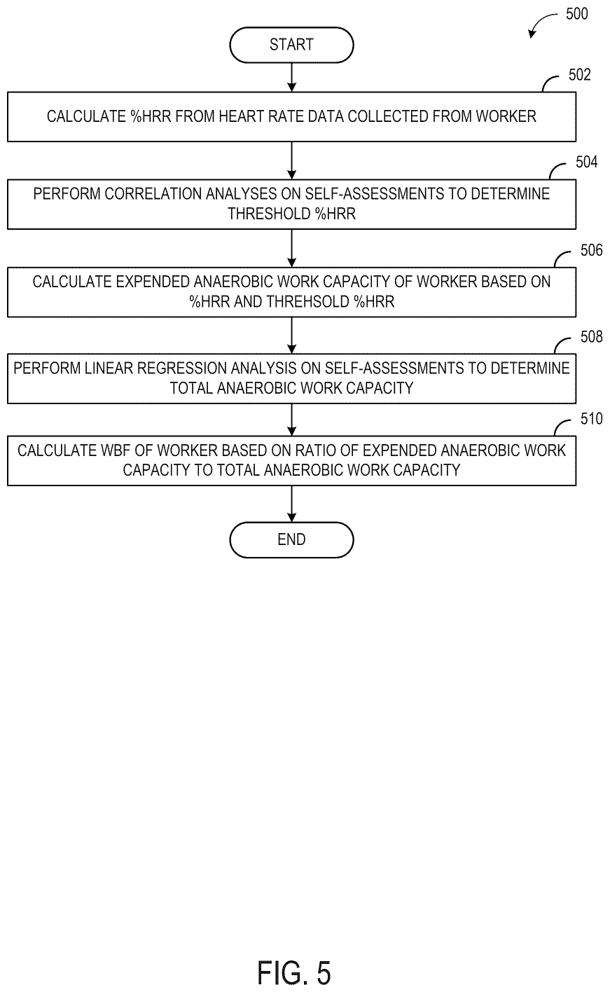

Additionally, during the physical activity, worker 101 may be requested by wearable WBF assessment device 150 to submit a WBF self-assessments 104 at periodic time intervals (e.g., 2-3 hours), where each WBF self-assessment 104 is a subjective, perceived degree of WBF by worker 101. WBF self-assessments 104 may be requested and submitted via WBF assessment device 150. The WBF self-assessments 104 may be compared to generate individual calibration data 106, which may be used to individually calibrate bioenergetic model 110 to worker 101. For example, various correlation analyses and/or linear regressions may be performed on WBF self-assessments 104 to generate individual calibration data 106, as described in greater detail below in reference to FIG. 5.

Referring to FIG. 2, a block diagram 200 shows an example of a WBF assessment device 202, in accordance with an embodiment, where WBF assessment device 202 may monitor the WBF of a worker during physical labor, as described above in reference to FIG. 1. WBF assessment device 202 may be a wearable device worn by the worker. In some embodiments, WBF assessment device 202 may be worn on a wrist of the worker, as described above. In other embodiments, WBF assessment device 202 may be worn at a different location on a body of the worker. In still other embodiments, one or more portions of WBF assessment device 202 may be worn on a body of the worker, and other portions of WBF assessment device 202 may not be worn on a body of the worker, where WBF assessment device 202 may be in wireless communication with a second device worn on the body of the worker, and the second device may transmit data collected from the worker via the second device (e.g., heart rate, etc.) to the WBF assessment device 202.

WBF assessment device 202 includes a processor 204 configured to execute machine readable instructions stored in non-transitory memory 206. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

WBF assessment device 202 includes a biosensor 216. WBF assessment device 202 may be worn by the worker such that biosensor 216 is in direct contact with a skin of the worker. For example, WBF assessment device 202 may be worn on the wrist of the worker, and biosensor 216 may be positioned on WBF assessment device 202 such that biosensor 216 is in face-sharing contact with skin on the wrist. As described above, biosensor 216 may be a photoplethysmographic sensor that acquires an optical signal by illuminating skin and detecting changes in absorbed or reflected light with a photodetector. The detected changes may be used to collect HR data from the worker, such as a resting HR, a maximum HR, and/or a % HRR of the worker.

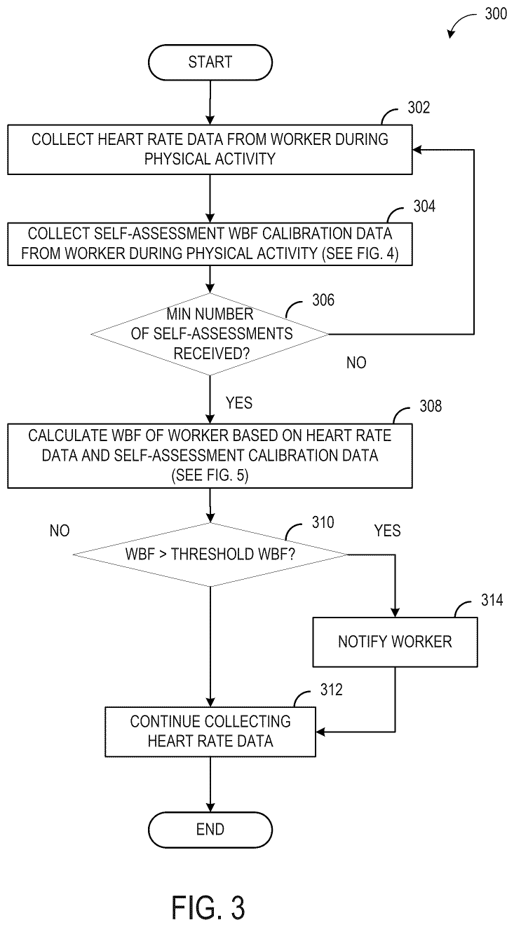

Non-transitory memory 206 may store a heart rate measurement module 208, which may store various programs for measuring and collecting HR data 210 via the biosensor, and for calculating the % HRR from the collected HR data. In particular, heart rate measurement module 208 may include instructions that when executed by processor 204 carry out one or more steps of method 300 of FIG. 3.

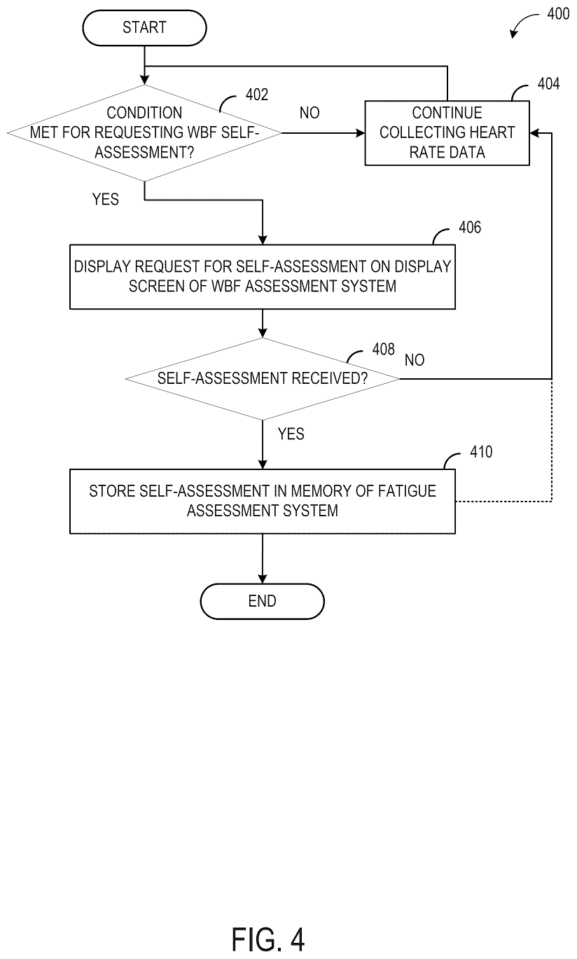

Non-transitory memory 206 may store a self-assessment module 212, which may store various programs for requesting self-assessments from the worker regarding the worker's perception of their WBF at a time when the self-assessments are requested. In particular, self-assessment module 212 may include instructions that when executed by processor 204 carry out one or more steps of method 400 of FIG. 4. Non-transitory memory 206 may store self-assessment data 214, which may include a plurality of self-assessments performed by the worker and submitted via WBF assessment device 202.

In various embodiments, the self-assessments may be performed and/or submitted via a set of user controls 220 and a display screen 218. For example, a request for a self-assessment may be displayed on display screen 218. Additionally or alternatively, the request may be indicated to the worker via an audio signal, or by a vibration of WBF assessment device 202. The self-assessment may rely on the worker selecting one or more of user controls 220. For example, the user controls 220 may be touchscreen controls on display screen 218, or buttons arranged on WBF assessment device 202, or a different kind of control element. The self-assessment may include one or more images displayed on display screen 218 that are edited, updated, or selected by the worker via user controls 220. The user controls 220 may include a submit button, for example, to submit a completed self-assessment. In one example, user controls 220 include one or more buttons that enable WBF assessment device 202 to begin monitoring the WBF of the worker and/or stop monitoring the WBF of the worker. The self-assessments are described in greater detail below in reference to FIG. 4.

It should be understood that WBF assessment device 202 shown in FIG. 2 is for illustration, not for limitation. Another appropriate WBF assessment device may include more, fewer, or different components.

Referring now to FIG. 3, an exemplary high-level method 300 is shown for using a WBF assessment device (e.g., WBF assessment device 202 of FIG. 2) to monitor a WBF of a worker performing physical labor, as described above in reference to FIG. 1. One or more instructions of method 300 may be executed by a processor of the WBF assessment device (e.g., processor 204) based on instructions stored in a non-transitory memory of the WBF assessment device (e.g., non-transitory memory 206).

Method 300 begins at 302, where method 300 includes collecting heart rate data from the worker while the worker is performing physical activity (e.g., physical labor). The physical activity may include tasks of varying physical intensity.

At 304, method 300 includes collecting self-assessment WBF calibration data from the worker during the physical activity. The self-assessment WBF calibration data may comprise a plurality of self-assessments of the worker's WBF, as perceived by the worker, using the WBF assessment device. For example, the worker may receive a request to perform a self-assessment on the WBF assessment device, and the worker may perform and submit the self-assessment using the WBF assessment device. The self-assessments are described in greater detail below in reference to FIG. 4.

At 306, method 300 includes determining whether a minimum number of self-assessments have been received. For example, the minimum number of self-assessments maybe one, two, or four, or six, or a different number of self-assessments. In some embodiments, the minimum number of self-assessments may be greater than one, such that statistical comparisons may be performed between different self-assessment.

If at 306 it is determined that the minimum number of self-assessments has not been received, method 300 proceeds back to 302, and method 300 continues collecting the heart rate data and the self-assessment WBF calibration data from the worker. Alternatively, if at 306 it is determined that the minimum number of self-assessments has been received, method 300 proceeds to 308.

At 308, method 300 includes calculating the WBF of the worker, based on the heart rate data at the self-assessment calibration data. In various embodiments, the WBF may be expressed as a percentage between 0 to 100%, with 0 indicating a lowest level of fatigue (e.g., no fatigue) and 100% indicating a highest level of fatigue (e.g., total exhaustion). The calculation of the WBF from the heart rate data on the self-assessment calibration data is described in greater detail below, in reference to FIG. 5.

At 310, after the WBF of the worker has been calculated, method 300 includes determining whether the calculated WBF of the worker is greater than a threshold WBF. In various embodiments, the threshold WBF may be determined by an employer or manager of the worker. The threshold WBF may be a level at which the worker is able to perform the various tasks comprised by the physical activity without endangering a health of the worker and/or other coworkers or individuals working in a vicinity of the worker. In some examples, the threshold WBF may be personalized to the worker. That is, a first threshold WBF may apply to a first worker; a second threshold WBF may apply to a second worker; a third threshold WBF may apply to a third worker; and so on. Additionally, a plurality of thresholds may be used for an individual. For example, at 50% fatigue, a worker may be advised to take a brief rest, whereas at 75%, the worker may be mandated to take a break.

If at 310 it is determined that the WBF is not greater than the threshold WBF, method 300 proceeds to 312. At 312, method 300 includes continuing to collect the heart rate data, and method 300 ends. In some embodiments, the WBF of the worker may be displayed to the worker on a display screen of the WBF assessment device (e.g., display screen 218 of FIG. 2), so that the worker is aware of their estimated level of whole body fatigue. Additionally or alternatively, the WBF of the worker may be stored in a memory of the WBF assessment device (e.g., non-transitory memory 206). Further, in some embodiments, the WBF of the worker may be transmitted wirelessly to a different device or system for further analysis and/or storage. For example, the WBF of the worker may be transmitted to a device of a manager of the worker who may be overseeing the physical activity of the worker, and/or to a computer system of an organization employing the worker that may monitor a WBF of various workers.

If at 310 it is determined that the WBF of the worker is greater than the threshold WBF, method 300 proceeds to 314. At 314, method 300 includes notifying the worker that the level of whole body fatigue of the worker has exceeded the threshold WBF for the worker. For example, an alert may be displayed on the display screen of the WBF assessment device. In some cases, the alert may be accompanied by sound and/or a vibration generated by the WBF assessment device. Additionally or alternatively, in some embodiments the WBF of the worker may be transmitted to the device and/or system of the manager and/or organization overseeing the physical activity of the worker. Based on the WBF, the manager may assign, reassign, or adjust a task being performed by the worker. For example, the manager may tell the worker to take a break or stop work until the WBF of the worker decreases below the threshold WBF. In other examples, the manager may tell the worker to stop performing a first task with a high PI, and begin work on a second task with a lower PI. In this way, the level of whole body fatigue of the worker may be monitored by the worker and/or the manager/organization to ensure that the worker does not suffer from exhaustion or other health risks, and to maintain a safe and healthy workplace.

After the worker is notified, method 300 may proceed to 312, where method 300 includes continuing to collect the heart rate data. Alternatively, in some embodiments, the heart rate data may not be collected after worker notification, and method 300 may end.

Referring now to FIG. 4, a method 400 is shown for collecting self-assessment data from a worker, such as worker 101 of FIG. 1, regarding a level of whole body fatigue of the worker while performing physical activity (e.g., physical labor). The self-assessment data (e.g., self-assessment data 214) may be collected and stored via a WBF assessment device, such as WBF assessment device 202 of FIG. 2. As described in greater detail below in reference to FIG. 5, the self-assessment data may be used to calibrate a bioenergetic model used to calculate the WBF of the worker. One or more instructions of method 400 may be executed by a processor of the WBF assessment device (e.g., processor 204) based on instructions stored in a non-transitory memory of the WBF assessment device, such as self-assessment module 212 of non-transitory memory 206 of FIG. 2.

Method 400 begins at 402, where method 400 includes determining whether a condition is met for requesting a WBF self-assessment from the worker. In various embodiments, the condition may include an elapsing of a predetermined time interval, where the predetermined time interval is established by an organization employing the worker, a manager of the worker, and/or the worker. For example, the organization may establish that the worker should perform a self-assessment of their WBF every two hours during the performance of the physical activity. In other examples, the time interval may be three hours, or one hour, or a different number of hours or minutes. In some examples, the time interval may be based on a number of self-assessments of the worker desired by the organization. For example, the organization may establish that six self-assessments of the worker daily over two eight-hour workdays are desirable to achieve an individual calibration of the bioenergetic model for the worker that generates a WBF assessment for the worker of a threshold quality and/or accuracy. For accurate calibration of the personalized WBF model, at least three self-assessments are required during a continuous recording duration, such as an 8-hour workday. Additionally, sufficient intervals should be maintained between each self-assessment, preferably at least 30 minutes. In other embodiments, a greater or lesser number of self-assessments of the worker may be desired, over a greater or lesser time period. In some examples, the organization may periodically request workers to complete self-assessments to maintain a threshold and/or accuracy. For instance, following the initial calibration, it might be beneficial to have a regular recalibration, perhaps every two days each month, to ensure consistent quality and accuracy.

In still other embodiments, WBF self-assessments of the worker may be requested at varying time intervals. For example, a first self-assessment may be requested after a first time interval; a second self-assessment may be requested after a second time interval, which may be different from the first time interval; a third self-assessment may be requested after a third time interval, which may be different from the first and/or second time intervals; and so on. Alternatively, in some embodiments, WBF self-assessments of the worker may not be requested at regular or irregular time intervals, and may be requested in response to a measured criterion of the worker, such as the worker's heart rate. For example, a calibration of the bioenergetic model may be desired that is specific to a range of heart rates.

If at 402 it is determined that the condition for requesting the WBF self-assessment has not been met, method 400 proceeds to 404. At 404, method 400 includes continuing to collect the heart rate data, and method 400 proceeds back to 402. Alternatively, if at 402 it is determined that the condition for requesting the WBF self-assessment has been met, method 400 proceeds to 406.

At 406, method 400 includes displaying a request for a self-assessment on a display screen of the WBF assessment device worn by the worker. In various embodiments, the worker may additionally or alternatively be notified of the request for the self-assessment by a vibration of the WBF assessment device and/or a sound generated by the WBF assessment device. When the worker receives the notification of the request, the worker may perform the self-assessment via the WBF assessment device.

The self-assessment may be minimally invasive and able to be completed with a short turnaround time for workers' responses. In various embodiments, a simple WBF self-report scale (e.g., Rating of Fatigue (ROF)) may be applied. For example, the ROF may be a pictographic single-item numerical scale (0-10) that workers can intuitively understand and reliably respond to in less than 15 seconds, even under dynamic work conditions in the field. In other words, the worker may be requested to rate their level of WBF on a scale of 1-10, using the pictographic. The ROF is different from alternative scales used to measure physical exertion, which have shown limitations in monitoring WBF in naturalistic work or daily-life environments when the job's physical demand is not consistent. ROF has been widely applied to monitor perceived WBF in not only exercise contexts but also during work and in daily life (e.g., construction sites and medical operating rooms) and found to correspond with the physiological changes closely correlated to WBF levels (e.g., blood lactate and respiratory exchange ratio).

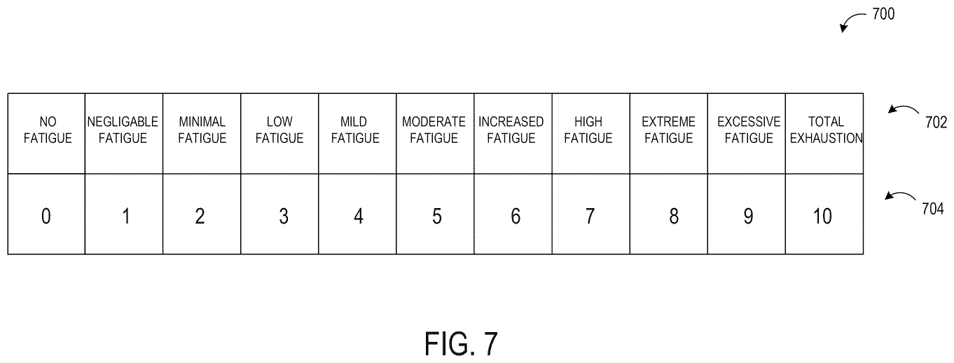

Referring briefly to FIG. 7, an exemplary pictograph 700 is shown, based on the numerical scale (0 to 10), where pictograph 700 may be used for a ROF of a WBF self-assessment. Pictograph 700 includes a 2×11 matrix of boxes in two rows. A first row 702 includes, within each box of first row 702, a description of a degree of fatigue, ranging from “NO FATIGUE” to “TOTAL EXHAUSTION”. A second row 704 includes, within each box of second row 704, a numerical scale of values from 0 to 10, such that each value included in a box of second row 704 is paired with a description in a corresponding box of first row 702. For example, 0 corresponds to no fatigue; 3 corresponds to low fatigue; 5 corresponds to moderate fatigue; 7 corresponds to high fatigue; and 10 corresponds to total exhaustion. Thus, using pictograph 700, a worker can quickly rate their fatigue in accordance with a standardized scale. The fatigue rating (e.g., the numerical value) can then be entered into the WBF assessment device.

Returning to FIG. 4, at 408, method 400 includes determining whether a self-assessment of the workers WBF has been received from the worker. In some cases, the self-assessment may not be received from the worker. The worker may be performing a task during which responding to the request for the self-assessment may not be feasible or safe, and may not perform the self-assessment. For example, the worker may be operating a vehicle, equipment, or machinery, or carrying a heavy object.

If at 408 it is determined that the requested self-assessment has not been received, method 400 proceeds back to 404, where method 400 includes continuing to collect the heart rate data. Alternatively, if at 408 it is determined that the requested self-assessment has been received, method 400 proceeds to 410. At 410, method 400 includes storing the self-assessment in a memory of the WBF assessment device (e.g., self-assessment data 214 of non-transitory memory 206 of FIG. 2).

After the self-assessment is stored in the memory, method 400 may end, or method 400 may proceed back to 404 to continue collecting the heart rate data.

Referring now to FIG. 5, a method 500 is shown for calculating a WBF of a worker, such as worker 101 of FIG. 1, while performing physical activity (e.g., physical labor), based on heart rate data collected via a WBF assessment device as described in method 300 and self-assessment calibration data collected via the WBF assessment device as described in method 400. One or more instructions of method 500 may be executed by a processor of the WBF assessment device (e.g., processor 204) based on instructions stored in a non-transitory memory of the WBF assessment device, such as non-transitory memory 206 of FIG. 2.

Before describing FIG. 5, a brief conceptual overview of the framework for determining the WBF of the worker should be provided. Method 500 relies on a bioenergetic model to determine the WBF of the worker. In the embodiment described below, a critical power (CP) model initially used in sports science to understand power intensity and exhaustion in single muscle groups is used. The CP model presumes two energy supplies: aerobic and anaerobic. Aerobic energy capacity [kJ] is theoretically unlimited but it has a rate limit based on oxygen delivery and capacity. In contrast, anaerobic work capacity [kJ] is limited by the amount of glycogen stored in the muscles and liver but is not rate limited. A CP threshold or CP represents a threshold PI, as a maximum sustainable aerobic capacity-based force rate without fatigue. When the worker's PI is smaller than CP (e.g., smaller than the threshold PI), sustained activity without fatigue is theoretically possible. When the worker's PI exceeds CP, anaerobic energy supply is used, depleting anaerobic work capacity and resulting in physical exhaustion. Thus, the level of WBF can be expressed as the proportion of expended anaerobic work capacity (AWCₑₓₚ [kJ]) over its total capacity (AWCₜₒₜ [kJ]):

$$WBF [ \% ] = {AWC}_{exp} / {AWC}_{tot} \times 100 \qquad ( 1 )$$

The variable AWCₑₓₚ can be calculated by integrating the difference between PI and CP over time. This integration can be represented as a recursive equation that accounts for the difference between PI and CP, as shown in Eq. (2). A max operator is used to ensure that AWCₑₓₚ does not decrease below zero. Using Eq. (2) along with Eq. (1), the workers' WBF can be continuously measured.

$${AWC}_{exp} ( t ) = max \{ 0 , {AWC}_{exp} ( t - 1 ) + [ ( PI ( t ) - CP ( t ) ) \times ( dt ) ] \} \qquad ( 2 )$$

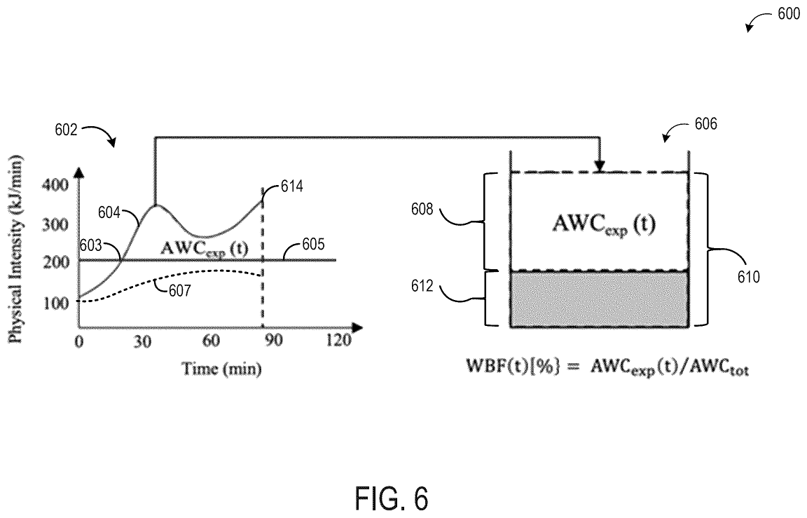

Referring briefly to FIG. 6, a CP diagram 600 is shown that illustrates how the WBF of the worker may be calculated. CP diagram 600 includes an exemplary PI graph 602 that plots a PI of work performed by the worker over time, indicated by line 604. A threshold PI (e.g., the CP) is indicated by a line 605, where the threshold PI is a level of physical intensity of the work above which the anaerobic work capacity of the worker begins to be depleted. Line 604 crosses the threshold PI indicated by line 605 at a point 603, after roughly 20 minutes. Subsequent work performed by the worker expends anaerobic work capacity (AWCₑₓₚ (t)), which generates a higher level of WBF over time until the worker reaches a point of exhaustion. For comparative purposes, second dotted line 607 indicates an alternative plot of the PI of the worker during a different set of tasks at a lower PI, that does not exceed the threshold PI indicated by line 605 (e.g., the CP). While performing the different set of tasks at the lower PI, the anaerobic work capacity of the worker is not depleted, whereby the WBF of the worker does not become higher over time until the worker becomes exhausted.

The expended anaerobic work capacity AWCₑₓₚ (t)) is represented by the area of graph 602 between line 604 and line 605, which can be quantified by integrating the difference between PI and CP over time as described above. CP diagram 600 includes a graphic 606 that shows a ratio of the expended anaerobic work capacity 608 (corresponding to the area of graph 602 between line 604 and line 605) to a total anaerobic work capacity 610 of the worker (AWCₜₒₜ), which represents a total capacity of the worker to perform work up to the point of total WBF (e.g., exhaustion). A portion 612 of the total anaerobic work capacity 610 of the worker corresponds to an amount of remaining anaerobic work capacity of the worker that could be applied to future physical activity. The worker's WBF is the ratio expressed as a percentage. For example in FIG. 6, the worker's WBF at a time indicated by dashed line 614 may be 65% (608/610×100).

Returning to FIG. 5, within this framework, method 500 begins at 502, where method 500 includes calculating a % HRR from the HR data collected from the worker during the physical activity. The HR data includes an HR of the worker, where in various embodiments, the HR is calculated from PPG data collected by a biosensor of the WBF assessment device as described above, using a Wiener filter-based algorithm. The HR may be calculated using a time window, such as a time window of 60 seconds.

The % HRR may be used as an individual PI index that tracks individual PI over time. In other words, % HRR is a relative measurement of PI generated by normalizing HR for individual differences. While % HRR does not provide separate PI for individual activities, gross PI estimated from % HRR offers a valuable clue for continuous monitoring of WBF, when combined with the CP model that integrates the difference between the gross PI and CP over time. As such, % HRR may be used as an indicator of workers' PI under diverse dynamic physical work environments. Specifically, % HRR and PI have a linear relationship within a range of 0%-65% HRR, covering most physical activities in occupational contexts.

% HRR assumes a resting HR (e.g., minimum HR during resting) as a level with no physical intensity, where a percentage of the difference between working and resting HRs among HR reserves is calculated as in Eq. (3) below.

$$\% HRR = ( {HR}_{working} - {HR}_{resting} ) / ( {HR}_{max} - {HR}_{resting} ) \qquad ( 3 )$$

Relative changes of HR due to physical activities are focused on to infer PI by offsetting each individual's different HR level due to their characteristics, such as hypertensive diseases and chronic mental stress. To convert HR to % HRR, the resting HR (HR_{resting}) can be measured during subjects' resting time. For example, the resting HR may be collected prior to the worker performing a task of the physical activity. The resting HR may also be assumed to be a lowest HR collected by the biosensor over a period during which the worker's WBF is being monitored by the WBF assessment device. In various embodiments, the worker's maximum HR (HRₘₐₓ) may be calculated as a function of an age of the worker. One example is shown in Eq. (4):

$${HR}_{max} [ beat / min ] = 208 - 0.7 \times Age [ yr ] \qquad ( 4 )$$

At 504, method 500 includes performing correlation analyses on WBF ratings included in the self-assessments submitted by the worker to determine a threshold % HRR of the worker. The threshold % HRR is the % HRR corresponding to CP (also referred to as % HRR_{CP}), which approximates the PI threshold indicated by line 505 of FIG. 5 described above.

When using the CP model to monitor fatigue or predict an exhaustion point of the worker, Eq. (1) and Eq. (2) are fed with PI measured in watts or in kJ/min by observing the physical activities and performance. Because the proposed technique measures % HRR as the workers' PI index, Eq. (1) and Eq. (2) is modified accordingly. Eq. (5) below describes a linear association between % HRR and PI by introducing a constant k [kJ/min % HRR] that indicates the unit PI per % HRR. Then, dividing both sides of Eq. (2) by k and changing CP/k to a new term % HRR_{CP} (e.g., % HRR at critical power) yields Eq. (6).

$$\% HRR ( t ) [ \% HRR ] = PI ( t ) [ k ] / min ] / k \qquad ( 5 )$$

$${AWC}_{exp} ( t ) / k = max \{ 0 , {AWC}_{exp} ( t - 1 ) / k + [ ( \% HRR ( t ) - \% {HRR}_{CP} ) \times ( dt ) ] \} \qquad ( 6 )$$

An appropriately determined % HRR_{CP} value will display a linear relationship between AWCₑₓₚ/k and WBF, as shown in Eq. (7) below. In one embodiment, the Pearson correlation coefficient between AWCₑₓₚ/k and a perceived WBF included in the self-assessment is examined by changing % HRR_{CP} from 0% HRR to 40% HRR in increments of 0.5% HRR. Then, the % HRR_{CP} value at the maximum Pearson correlation coefficient is determined as a worker's individual % HRR_{CP}.

At 506, method 500 includes calculating an expended an aerobic work capacity AWCₑₓₚ (t) of the worker based on the continuously tracked % HRR and the threshold % HRR (% HRR_{CP}), using Eq. (6) above.

At 508, method 500 includes performing a linear regression analysis on the self-assessments to determine a total anaerobic work capacity of the worker (AWCₜₒₜ). In one example, the value of k/AWCₜₒₜ is determined by conducting a linear regression analysis using at least six WBF ratings collected in at least six self-assessments over two 8 hour workdays. In this model, the self-assessment values serve as the independent variable, while AWCₑₓₚ at a certain time point acts as the predictor. The resulting coefficient from the regression analysis is represented as k/AWCₜₒₜ.

At 510, method 500 includes calculating a WBF of the worker based on a ratio of the expended anaerobic work capacity to the total anaerobic work capacity, using Eq. (7), which is generated by dividing the right side of Eq. (1) by k and multiplying it again. The WBF is expressed as a percentage by multiplying by 100:

$$WBF ( t ) [ \% ] = ( {AWC}_{exp} / k ) ( t ) \times k / {AWC}_{tot} \times 100 \qquad ( 7 )$$

The calculated WBF may then be compared to a threshold WBF, as described in method 300 in reference to FIG. 3. If the calculated WBF exceeds the threshold WBF, the worker and/or a manager/employer of the worker may be notified to prevent the worker from reaching the point of exhaustion.

In this way, % HRR and % HRR_{CP} are substituted for PI to use the CP bioenergetic model to calculate the WBF of the worker. Additionally, the CP model is calibrated using perceived WBF ratings included in the self-assessments periodically performed by the worker. By calibrating the CP model based on the self-assessments, an accuracy of the calculated WBF may be increased for each individual worker. As a result, the proposed monitoring technique is able to predict workers' WBF during daily work at actual jobsites, in contrast to other approaches. Unlike the localized muscle fatigue, WBF involves central fatigue factors impacting cognitive capabilities, thereby negatively affecting productivity and safety. Continuous monitoring empowers interventions to maintain appropriate WBF levels. For workers exhibiting high WBF, individual interventions such as work skill training, habit consultations, and nutrition guidance can be implemented. Task-wise observation of WBF enables the identification of physically demanding tasks, allowing for task-level interventions such as task redesign or schedule adjustments to reduce physical burden. An additional advantage of this monitoring technique is that it may facilitate future studies to assess a direct impact of WBF on injury risk, which could inform risk management programs with threshold limit values.

Although alternative wearable biosensor-based techniques have been introduced to monitor WBF in the field, the techniques used may actually monitor whole-body physical exertion, rather than WBF. Because physical exertion and WBF are not consistently correlated in a naturalistic work environment due to physical demand alteration, the performance of existing techniques in the field cannot be guaranteed. In contrast, the disclosed WBF monitoring technique applies the critical power (CP) bioenergetic model, modified to take % HRR observed using a wrist-worn PPG sensor as an index of physical intensity. Given that the proposed technique can be applied in the workplace with an affordable biosensor and a simple, minimally-invasive customization process based on worker self-assessments, this technique has potential to significantly help manage workers' WBF in the field, thereby contributing to the workplace safety and productivity.

The technical effect of customizing a bioenergetic model used to calculate a WBF of a worker based on periodic WBF self-assessments performed by the worker in the field using a wrist-worn WBF assessment device is that the worker's WBF may be more effectively managed to increase the productivity and health of the worker and increase workplace safety. Further, the system may provide increased processing because the bioenergetic model uses the WBF, rather than hear rate data directly, which can be more efficiently correlated to the individual worker via the self-assessments. Further, because the self-assessment data comes only at unexpected conditions where the worker is performing physical activity, the approach described herein is better able to provide consistent WBF assessments by using the bioenergetics model, rather than directly using heart rate data. In this way, more accurate assessments are provided with more efficient processor usage, leading to a synergistic result that also solves the technical problem of reducing data storage since the model, rather than the heart rate data directly, is used.

The disclosure also provides support for a method for a WBF assessment device used to assess a whole-body fatigue (WBF) of a worker, the method comprising: collecting heart rate data of the worker via a biosensor of the WBF assessment device, estimating the WBF of the worker based on the collected heart rate data, using a bioenergetic model, and in response to the WBF exceeding a threshold WBF, notifying the worker, wherein the bioenergetic model is individually calibrated to the worker based on a plurality of WBF self-assessments performed by the worker via the WBF assessment device at time intervals while the worker is performing physical activity. In a first example of the method, the biosensor is photoplethysmogram (PPG) sensor worn on a body of the worker. In a second example of the method, optionally including the first example, estimating the WBF of the worker based on the heart rate using the bioenergetic model further comprises: calculating a percentage of heart rate reserve (% HRR) of the worker based on the heart rate data, estimating a physical intensity (PI) of work performed by the worker, based on the % HRR, estimating a critical power (CP) threshold of the worker, the CP a maximum sustainable aerobic capacity-based force rate of the worker without fatigue, determining a % HRR at the CP threshold of the worker (% HRRCP) and a total anaerobic work capacity of the worker based on the plurality of WBF self-assessments performed by the worker, integrating a difference between the estimated PI and the % HRRCP over time to calculate an expended anaerobic work capacity of the worker, and estimating the WBF as a proportion of expended anaerobic work capacity of the worker over the total anaerobic work capacity of the worker. In a third example of the method, optionally including one or both of the first and second examples, determining the % HRRCP and the total anaerobic work capacity of the worker based on the plurality of WBF self-assessments performed by the worker further comprises: performing a series of correlation analyses on the plurality of WBF self-assessments performed by the worker to determine the % HRRCP, and performing a linear regression analysis on the plurality of WBF self-assessments performed by the worker to determine the total anaerobic work capacity. In a fourth example of the method, optionally including one or more or each of the first through third examples, performing a series of correlation analyses on the plurality of WBF self-assessments performed by the worker to determine the % HRRCP of the worker further comprises: examining a Pearson correlation coefficient between the expended anaerobic work capacity and a self-assessed WBF by changing the % HRRCP from 0% HRR to 40% HRR if increments of 0.5% HRR, and selecting the % HRRCP at the maximum Pearson correlation coefficient. In a fifth example of the method, optionally including one or more or each of the first through fourth examples, the plurality of WBF self-assessments performed by the worker comprises at least six self-assessments during two 8 hour workdays. In a sixth example of the method, optionally including one or more or each of the first through fifth examples, the regular time intervals are between two and three hours. In a seventh example of the method, optionally including one or more or each of the first through sixth examples, the WBF assessment device is a wearable device worn on a wrist of the worker. In a eighth example of the method, optionally including one or more or each of the first through seventh examples, each self-assessment of the plurality of WBF self-assessments performed by the worker includes a rating of fatigue (ROF) based on a pictographic single-item numerical scale that can be performed by the worker via the WBF assessment device in less than 15 seconds during the physical activity. In a ninth example of the method, optionally including one or more or each of the first through eighth examples, the method further comprises: at each time interval of the time intervals: displaying the ROF to the worker via a screen of the WBF assessment device, receiving an input from the worker via a user control of the WBF assessment device, the input including the self-assessment. In a tenth example of the method, optionally including one or more or each of the first through ninth examples, notifying the worker further comprises at least one of: displaying an alert on the screen of the WBF assessment device, playing a sound via the WBF assessment device, and generating a vibration at the WBF assessment device.

The disclosure also provides support for a wearable whole body fatigue (WBF) assessment device for determining a WBF of a worker, the wearable WBF assessment device comprising: a biosensor in contact with a skin of the worker, a processor, and a memory including instructions that when executed, cause the processor to: monitor a heart rate of the worker via the biosensor, estimate the WBF of the worker based on the measured heart rate, using a bioenergetic model calibrated to the worker, and in response to the WBF exceeding a threshold WBF, notify the worker. In a first example of the system, the memory includes further instructions that when executed, cause the processor to: notify the worker to perform self-assessments of the WBF of the worker via a display screen of the wearable WBF assessment device at a plurality of time intervals, and calibrate the bioenergetic model based on the self-assessments. In a second example of the system, optionally including the first example, the memory includes further instructions that when executed, cause the processor to: calculate a percentage of heart rate reserve (% HRR) of the worker based on the measured heart rate, estimate a physical intensity (PI) of work performed by the worker, based on the % HRR, perform a series of correlation analyses on the self-assessments to determine a threshold % HRR of the worker, calculate an expended anaerobic work capacity of the worker based on the estimated PI and the threshold % HRR, perform a linear regression analysis on the self-assessments to determine a total anaerobic work capacity of the worker, calculate the WBF based on a ratio of the calculated expended anaerobic work capacity of the worker to the total anaerobic work capacity of the worker. In a third example of the system, optionally including one or both of the first and second examples, each self-assessment of the self-assessments of the WBF includes a rating of fatigue (ROF) based on a pictographic single-item numerical scale that can be performed by the worker via the display screen during performing of work by the worker. In a fourth example of the system, optionally including one or more or each of the first through third examples, the memory includes further instructions that when executed, cause the processor to notify the worker by at least one of: displaying an alert on the display screen, playing a sound, and generating a vibration of the wearable WBF assessment device. In a fifth example of the system, optionally including one or more or each of the first through fourth examples, the wearable WBF assessment device is worn on a wrist of the worker.

The disclosure also provides support for a method for a wearable WBF assessment device worn by a worker while performing work, the method comprising: collecting heart rate data of the worker via a biosensor of the wearable WBF assessment device, displaying a request to the worker on a screen of the wearable WBF assessment device for the worker to perform a self-assessment of a whole body fatigue (WBF) of the worker, at regular time intervals, in response to receiving a plurality of self-assessments from the worker via an input device of the wearable WBF assessment device, calibrating a bioenergetic model of the wearable WBF assessment device, using the bioenergetic model to estimate the WBF of the worker, and in response to the WBF exceeding a threshold WBF, notifying the worker. In a first example of the method, using the bioenergetic model to estimate the WBF of the worker further comprises: calculating a percentage of heart rate reserve (% HRR) of the worker based on the heart rate data, estimating a physical intensity (PI) of work performed by the worker, based on the % HRR, calculating an expended anaerobic work capacity of the worker based on the estimated PI and a threshold % HRR, and calculating the WBF based on a ratio of the calculated expended anaerobic work capacity of the worker to a total anaerobic work capacity of the worker, wherein the threshold % HRR and the total anaerobic work capacity are calculated based on one or more self-assessments received during calibration of the bioenergetic model. In a second example of the method, optionally including the first example, the one or more self-assessments include a rating of fatigue (ROF) based on a pictographic single-item numerical scale that can be performed by the worker via a display screen of the wearable WBF assessment device during performing of work by the worker.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a WBF assessment device used to assess a whole-body fatigue (WBF) of a worker, the method comprising:
collecting heart rate data of the worker via a biosensor of the WBF assessment device;
estimating the WBF of the worker based on the collected heart rate data, using a bioenergetic model; and
in response to the WBF exceeding a threshold WBF, notifying the worker;
wherein the bioenergetic model is individually calibrated to the worker based on a plurality of WBF self-assessments performed by the worker via the WBF assessment device at time intervals while the worker is performing physical activity.

2. The method of claim 1, wherein the biosensor is a photoplethysmogram (PPG) sensor worn on a body of the worker.

3. The method of claim 1, wherein estimating the WBF of the worker based on the heart rate using the bioenergetic model further comprises:
calculating a percentage of heart rate reserve (% HRR) of the worker based on the heart rate data;
estimating a physical intensity (PI) of work performed by the worker, based on the % HRR;
estimating a critical power (CP) threshold of the worker, the CP a maximum sustainable aerobic capacity-based force rate of the worker without fatigue;
determining a % HRR at the CP threshold of the worker (% HRR_{CP}) and a total anaerobic work capacity of the worker based on the plurality of WBF self-assessments performed by the worker;
integrating a difference between the estimated PI and the % HRR_{CP} over time to calculate an expended anaerobic work capacity of the worker; and
estimating the WBF as a proportion of expended anaerobic work capacity of the worker over the total anaerobic work capacity of the worker.

4. The method of claim 3, wherein determining the % HRR_{CP} and the total anaerobic work capacity of the worker based on the plurality of WBF self-assessments performed by the worker further comprises:
performing a series of correlation analyses on the plurality of WBF self-assessments performed by the worker to determine the % HRR_{CP}; and
performing a linear regression analysis on the plurality of WBF self-assessments performed by the worker to determine the total anaerobic work capacity.

5. The method of claim 4, wherein performing a series of correlation analyses on the plurality of WBF self-assessments performed by the worker to determine the % HRR_{CP} of the worker further comprises:
examining a Pearson correlation coefficient between the expended anaerobic work capacity and a self-assessed WBF by changing the % HRR_{CP} from 0% HRR to 40% HRR if increments of 0.5% HRR; and
selecting the % HRR_{CP} at the maximum Pearson correlation coefficient.

6. The method of claim 1, wherein the plurality of WBF self-assessments performed by the worker comprises at least six self-assessments during two 8-hour workdays.

7. The method of claim 1, wherein the regular time intervals are between two and three hours.

8. The method of claim 1, wherein the WBF assessment device is a wearable device worn on a wrist of the worker.

9. The method of claim 1, wherein each self-assessment of the plurality of WBF self-assessments performed by the worker includes a rating of fatigue (ROF) based on a pictographic single-item numerical scale that can be performed by the worker via the WBF assessment device in less than 15 seconds during the physical activity.

10. The method of claim 9, further comprising:
at each time interval of the time intervals:
displaying the ROF to the worker via a screen of the WBF assessment device;
receiving an input from the worker via a user control of the WBF assessment device, the input including the self-assessment.

11. The method of claim 10, wherein notifying the worker further comprises at least one of:
displaying an alert on the screen of the WBF assessment device;
playing a sound via the WBF assessment device; and
generating a vibration at the WBF assessment device.

12. A wearable whole body fatigue (WBF) assessment device for determining a WBF of a worker, the wearable WBF assessment device comprising:
a biosensor in contact with a skin of the worker;
a processor; and
a memory including instructions that when executed, cause the processor to:
monitor a heart rate of the worker via the biosensor;
estimate the WBF of the worker based on the measured heart rate, using a bioenergetic model calibrated to the worker; and
in response to the WBF exceeding a threshold WBF, notify the worker.

13. The wearable WBF assessment device of claim 12, wherein the memory includes further instructions that when executed, cause the processor to:
notify the worker to perform self-assessments of the WBF of the worker via a display screen of the wearable WBF assessment device at a plurality of time intervals; and
calibrate the bioenergetic model based on the self-assessments.

14. The wearable WBF assessment device of claim 13, wherein the memory includes further instructions that when executed, cause the processor to:
calculate a percentage of heart rate reserve (% HRR) of the worker based on the measured heart rate;
estimate a physical intensity (PI) of work performed by the worker, based on the % HRR;
perform a series of correlation analyses on the self-assessments to determine a threshold % HRR of the worker;
calculate an expended anaerobic work capacity of the worker based on the estimated PI and the threshold % HRR;
perform a linear regression analysis on the self-assessments to determine a total anaerobic work capacity of the worker;
calculate the WBF based on a ratio of the calculated expended anaerobic work capacity of the worker to the total anaerobic work capacity of the worker.

15. The wearable WBF assessment device of claim 13, wherein each self-assessment of the self-assessments of the WBF includes a rating of fatigue (ROF) based on a pictographic single-item numerical scale that can be performed by the worker via the display screen during performing of work by the worker.

16. The wearable WBF assessment device of claim 13, wherein the memory includes further instructions that when executed, cause the processor to notify the worker by at least one of:
displaying an alert on the display screen;
playing a sound; and
generating a vibration of the wearable WBF assessment device.

17. The wearable WBF assessment device of claim 12, wherein the wearable WBF assessment device is worn on a wrist of the worker.

18. A method for a wearable WBF assessment device worn by a worker while performing work, the method comprising:
collecting heart rate data of the worker via a biosensor of the wearable WBF assessment device;
displaying a request to the worker on a screen of the wearable WBF assessment device for the worker to perform a self-assessment of a whole body fatigue (WBF) of the worker, at regular time intervals;
in response to receiving a plurality of self-assessments from the worker via an input device of the wearable WBF assessment device, calibrating a bioenergetic model of the wearable WBF assessment device;
using the bioenergetic model to estimate the WBF of the worker; and
in response to the WBF exceeding a threshold WBF, notifying the worker.

19. The method of claim 18, wherein using the bioenergetic model to estimate the WBF of the worker further comprises:
calculating a percentage of heart rate reserve (% HRR) of the worker based on the heart rate data;
estimating a physical intensity (PI) of work performed by the worker, based on the % HRR;
calculating an expended anaerobic work capacity of the worker based on the estimated PI and a threshold % HRR; and
calculating the WBF based on a ratio of the calculated expended anaerobic work capacity of the worker to a total anaerobic work capacity of the worker;
wherein the threshold % HRR and the total anaerobic work capacity are calculated based on one or more self-assessments received during calibration of the bioenergetic model.

20. The method of claim 18, wherein the one or more self-assessments include a rating of fatigue (ROF) based on a pictographic single-item numerical scale that can be performed by the worker via a display screen of the wearable WBF assessment device during performing of work by the worker.
